(19) United States
(12) Patent Application Publication
(10) Pub. No.: US 2009/0318558 A1
(43) Pub. Date: Dec. 24, 2009

(54) SOLVENT SYSTEM OF HARDLY SOLUBLE DRUG WITH IMPROVED DISSOLUTION RATE

(76) Inventors: Jae-Hwan Kim, Seoul (KR); Kyung-Sik Lee, Suwon-shi (KR); Woo-Choul Shin, Suwon-shi (KR); So-Ra Lee, Seoul (KR); Jae-Hun Yi, Suwon-shi (KR)

Correspondence Address: SMITH PATENT OFFICE, 1901 PENNSYLVANIA AVENUE N W, SUITE 901, WASHINGTON, DC, 20006, US

(21) Appl. No.: 12/486,608

(22) Filed: Jun. 17, 2009

Related U.S. Application Data
(63) Continuation-in-part of application No. 10/682,989, filed on Oct. 14, 2003, now abandoned.

(30) Foreign Application Priority Data
Feb. 12, 2003 (KR) 10-2003-0008931
Feb. 21, 2003 (KR) 10-2003-0011056
Sep. 1, 2003 (KR) 1020030060665

(51) Int. Cl. A61K 31/192; A61P 29/00

(52) U.S. Cl. 514/569; 514/570

(57) ABSTRACT

The present invention relates to a solvent system with improved disintegration degree and dissolution ratio of a hardly soluble drug by highly concentrating the drug through partial ionization, and by establishing optimal conditions for enhancing bioavailability of the drug, such as the co-relation between the acid drug and the accompanied components, ionization degree of a solvent system, use of an appropriate cation acceptor, water content, selection of optimal mixing ratio of the respective components and use of specific surfactants, and to a pharmaceutical preparation comprising the same. The solvent system of the invention has advantages in that it can enhance bioavailability by improving the disintegration degree and dissolution ratio of a hardly soluble drug and also provide a capsule with a sufficiently small volume to permit easy swallowing.

### CROSS REFERENCE TO RELATED APPLICATIONS

This application is a continuation-in-part application of co-pending U.S. patent application Ser. No. 10/682,989 filed on Oct. 14, 2003, currently pending. U.S. patent application Ser. No. 10/682,989 is incorporated herein by reference.

### BACKGROUND OF THE INVENTION

1. Field of the Invention

The present invention relates to a solvent system with improved disintegration degree and dissolution ratio of a hardly soluble drug by highly concentrating the drug through partial ionization, and by establishing optimal conditions for enhancing bioavailability of the drug, such as the co-relation between the acid drug and the accompanied components, ionization degree of a solvent system, use of an appropriate cation acceptor, water content, selection of optimal mixing ratio of the respective components and use of specific surfactants, and to a pharmaceutical preparation comprising the same.

2. Discussion of the Related Art

In general, not all liquids are suitable as a vehicle or carrier for the filling material encapsulated in a soft capsule. For example, liquid is an indispensable part for the filling material of a capsule. However, water miscible liquids and volatile liquids cannot be contained as one of major components of the capsule filling materials since they can be migrated to the hydrophilic gelatin shell or penetrated through the gelatin shell to be volatilized. Such examples include water, alcohols, as well as emulsions. Similarly, gelatin plasticizers such as glycerin and propylene glycol cannot be a major component of the capsule filling material since the gelatin shell is highly susceptible of heat and humidity. However, water and alcohols can be used as a subsidiary component (less than about 5% of the capsule filling material), for example, a dissolution aid upon preparation of the capsule filling solution. Also, glycerin or propylene glycol in an amount of less than 10% can be used as a co-solvent, along with a liquid such as polyethylene glycol to cure the shell. Liquids which are widely used in the preparation determination include oil phases such as vegetable oils, mineral oils, non-ionic surfactants, polyethylene glycol (400, 600) and the like, which can be used alone or in combination.

All the liquids, solutions, suspensions for preparation of capsules should be homologues and free-bubbles, and can flow by themselves at a temperature not exceeding 35. This is because the adhesion temperature of the gelatin shell is 37 to 40° C. Also, the preparation to be formulated has a pH of 2.0 to 8.0. If the pH of the preparation is more acidic than the lower limit, hydrolysis may occur to weaken the gelatin shell, causing leakage. If the filling material is basic, the gelatin shell is tanned to induce cross-linking in the gelatin shell, which delays the disintegration time of the soft capsule.

Upon investigation of prior arts related to soft capsules, U.S. Pat. No. 3,557,280 (Jan. 19, 1971) discloses the preparation of aqueous solutions of oxytetracycline. Specifically, pH was adjusted to the range of 8.0 to 9.5 to increase the storage life span of a hardly soluble drug and magnesium hydroxide (Mg(OH)₂) is used to increase the solubility of the filling material. However, when a gelatin capsule is prepared according to this prescription, cross linkings may occur within the gelatin molecular, causing the capsule shell insoluble, which is not proper for the object of the present invention.

Korean Patent No. 10-0185294 disclosed a method for producing an Ibuprofen composition comprising Ibuprofen, polyvinylpyrrolidone and polyethylene glycol, in which combined surfactants (polyoxyethylene sorbitan fatty acid ester and polyoxy 40 castor oil) are added to a solution heated to 40 to 50° C. This invention is similar to the present invention in that a combination of surfactants is used to improve the dissolution rate and the bioavailability of Ibuprofen. However, problems of highly hardly soluble drugs such as Naproxen cannot be solved by the simple use of a surfactant.

Also, an example of the conventional ionizable solvent system is disclosed in U.S. Pat. No. 5,071,643. The object of this invention is for increasing the solubility of a drug by partial ionization. The use of sodium hydroxide (NaOH) accords with the object of the present invention in one aspect. However, strictly speaking, this invention is limited to a step to dissolvate a hardly soluble drug in an ionizable pharmaceutical solvent system by depending on pH only and the system has problems of precipitation as time goes by. Also, it teaches the use of glycerin or polyvinylpyrrolidone (Povidon) to increase an amount of a drug capable of being dissolved in a given volume of a liquid. However, it aims only at increase of the solubility in a prescribed volume.

Also, U.S. Pat. No. 4,002,718 discloses use of polyvinylpyrrolidone or glycerin in a small amount to hasten dissolution of micronized Digoxin in polyethylene glycol in the preparation of a solution suitable for a soft gel.

According to the foregoing prior arts, it has been described that an extremely diluted solution (0.1%) and glycerin, propylene glycol, or polyvinylpyrrolidone (Povidon) are used in the preparation of a highly concentrated solution for producing a capsule, but there is no description regarding the improvements of the disintegration and dissolution rate. Also, since the capsules prepared according to the prior arts has an extremely low dissolution rate or the preparation are too bulky, and thus the arts failed to realize products in a commercial level.

### SUMMARY OF THE INVENTION

The present inventors have conducted researches and studies to seek a method for improving bioavailability of hardly soluble drugs, and as a results, discovered that the bioavailability of the drugs can significantly be improved by highly concentrating the drug through partial ionization, and by compositely establishing optimal conditions for enhancing bioavailability of the drug, such as the co-relation between the acid drug and the accompanied components, ionization degree of a solvent system, use of an appropriate cation acceptor, water content, selection of optimal mixing ratio of the respective components and use of specific surfactants, and completed the present invention.

Thus, it is a primary object of the present invention to provide a solvent system which can prepare a highly concentrated solution of a hardly soluble drug or acidic drug and it is another object of the present invention to provide preparations such as a soft capsule having the improved disintegration degree and dissolution rate improved while having the bioavailability increased by the highly concentrated dissolution.

It is yet another object of the present invention to provide a pharmaceutical formulation, for example, a soft capsule, two-piece capsule or tablet comprising the above solvent system.

To achieve the above object, in an aspect of the present invention, there is provided a solvent system for a hardly soluble drug or an acidic drug having the improved disintegration and dissolution rates, whereby the effect of the drug, that is, the bioavailability which is the ultimate purpose of a preparation, is improved, and a pharmaceutical preparation comprising the solvent system and a hardly soluble acidic drug.

More particularly, the pharmaceutical preparation according to the present invention comprises a hardly soluble acidic drug and a solvent system therefor, in which the solvent system comprises a pharmaceutically acceptable cation acceptor for increasing the solubility of the drug by partially ionizing the drug so that the drug exists in two forms of a free acid and a cationic salt, polyethylene glycol, water and a surfactant to improve the dissolution rate.

In a preferred embodiment according to the present invention, the solvent system comprises 10 to 90% by weight, preferably 10 to 80% by weight, more preferably 30 to 70% by weight of polyethylene glycol, 0.1 to 50% by weight, preferably 0.2 to 40% by weight, more preferably 0.2 to 30% by weight of a surfactant and 1 to 15% by weight, preferably 3 to 12% by weight, more preferably 4 to 9% by weight of water, and 0.1 to 2 mole equivalent of a cation acceptor with respect to the hardly soluble acidic drug.

As an exemplary to help better understanding of the present invention, the solvent system simply comprises 10 to 90% by weight of polyethylene glycol (more preferably, polyethylene glycol 600), 0.1 to 2 mole equivalent of a cation acceptor (more preferably KOH, NaOH) per mole equivalent of the hardly soluble acidic drug to increase the solubility of the hardly soluble acidic drug, 0.1 to 50% by weight of a vehicle selected from surfactants (more preferably, Polyoxy 40 hydrogenated castor oil) and 0.1 to 15% by weight of water.

### BRIEF DESCRIPTION OF THE DRAWINGS

The above and other objects, features and advantages of the present invention will be apparent from the following detailed description of the preferred embodiments of the invention in conjunction with the accompanying drawing, in which:

FIG. 1 is a graph showing the relation between the dissociation and the ionization of a drug with carboxylic acid, in which the ionization was performed using 10% KOH solution at 105° C. for one week (Y: dissociation rate, X: ionization degree);

FIG. 2 is a graph showing the dissolution rate of a prescription according to the present invention and a comparative prescription in water;

FIG. 3 is a graph showing the dissolution rate of a prescription according to the present invention and a comparative prescription in a phosphate buffer (pH 7.4);

FIG. 4 is a graph showing the dissolution rate of a prescription according to the present invention and a comparative prescription at pH 1.2;

FIG. 5 is a graph showing the dissolution rate of a prescription according to the present invention and a comparative prescription at pH 4.0;

FIG. 6 is a graph showing the dissolution rate of a prescription according to the present invention and a comparative prescription at pH 6.8;

FIG. 7 is a graph showing the dissolution rate of a prescription according to the present invention and another comparative prescription in water;

FIG. 8 is a graph showing the dissolution rate of a prescription according to the present invention and another comparative prescription in water;

FIG. 9 is a graph showing the dissolution rate of the prescription according to the present invention and another comparative prescription at pH 6.8; and

FIG. 10 is a graph showing the dissolution rate of the prescription according to the present invention and a comparative prescription at pH 1.2.

### DETAILED DESCRIPTION OF THE PREFERRED EMBODIMENT

Hereinafter, the present invention is described in detail.

The solvent system according to the present invention is anew one with the improved disintegration and dissolution rate by adding a specific surfactant or a cation acceptor to a highly concentrated capsule filling material, which does not cause the precipitation problem even after time goes by, and thus can be used to prepare a highly concentrated solution of a hardly soluble drug.

The solvent system of the invention primarily increases the solubility of a hardly soluble drug capable of being partially ionized to form a highly concentrated solution and secondarily improves the disintegration and the dissolution rates. Therefore, even when a liquid filling material is encapsulated in a soft capsule, the solvent system can improve the disintegration and dissolution rates of the capsule filling material. Also, the solvent system is very useful in that it can effectively encapsulate a drug in a highly concentrated solution with a volume that is small enough to permit easy swallowing.

The conventional approaches to improve the availability of hardly soluble drugs have widely been carried out depending on the selection of a surfactant type vehicle and in some cases, unexpectedly, significant results were observed even when well known components were applied. Some of the previously mentioned prior arts are the cases. However, hardly soluble drugs have different solubility according to their chemical properties and the simple selection of a certain surfactant cannot be generally applied to drugs with extremely low solubility (for example, Naproxen).

The system according to the present invention is definitely distinguished from the conventional systems in that it can be generally applied to hardly soluble compounds with extremely low solubility. For example, Ibuprofen is well soluble in ethanol, acetone, and chloroform but hardly soluble in water while Naproxen is well soluble in acetone, soluble in chloroform but hardly soluble in water. That is, Ibuprofen can be solvated and formulated using common vehicles in some cases because it is better soluble than Naproxen for many vehicles, and also has a low melting point. However, in case of drugs with an extremely low solubility such as Naproxen, it has been impossible to be effectively dissolved until the present invention.

Therefore, the present invention employs a solvent system which is definitely distinguishable from the prior arts, which is accomplished by compositely considering various factors, including optimal conditions for enhancing bioavailability of hardly water-soluble acidic drugs, that is, the relation between the hardly water-soluble acidic drugs and each of accompanied components, ionization degree of the solvent system, use of an appropriate cationic acceptor, water content and selection of the optimal mixing ratio of the constituting components, and therefore any of prior arts does not teach the present invention in this point of view. The hardly water-soluble acidic drug as used herein is meant to indicate the acidic drag which has low or extremely low water-solubility and has a solubility in water at 25° C. of 10 mg/ml or less, can interchangeably be used as “poorly water-soluble acidic drug” or “sparingly water-soluble acidic drug (see U.S. Pat. No. 6,231,890), and includes representative examples of the acidic drugs as discussed later.

The improvement of the bioavailability which is sought in the present invention can be accomplished when the ionization degree of the drug reaches in the range of 10% to 65%, more preferably 40 to 55%, most preferably about 50% and the water content in the solvent system is less than 15%, in addition to use of the specific surfactant(s).

Moreover, by establishing the above-described conditions, the present invention has advantages in that it can provide further benefits in addition to the improvement of the disintegration rate and the dissolution rate of a highly concentrated solution; that is, surfactants with various advantageous properties can be used alone or in combination, a capsule shell can be produced without glycerin, and the products made by encapsulating a drug in a highly concentrated solution by using the solvent system of the present invention has a relatively small volume allowing easy swallowing, as compared to products made by encapsulating a drug according to the conventional dissolution method. Specifically, if 200 mg of Ibuprofen is formulated in a soft capsule using the solvent system according to the present invention, it is possible to reduce the capsule filling material as small as 516 mg. However, if 200 mg of Ibuprofen is formulated according to the prior arts, it is difficult to expect improvement in the dissolution rate even though it is formulated in an amount less than 600 mg. As another example, if 250 mg of Naproxen is formulated by applying the system of the present invention, a capsule can be made to contain the capsule filling material in an amount of 800 mg. However, it is difficult to formulate a capsule with a volume of less than 1400 mg since the dissolution rate is significantly low.

Therefore, the pharmaceutical preparation according to the present invention and a solvent system therefor has the characteristics that the dissolution and disintegration rates and the bioavailability are improved, and a small-sized capsule that can be readily taken by a consumer was formulated for the first time.

The representative examples of the acidic drugs which can be applied to the solvent system according to the present invention include naproxen (C₁₄H₁₄O₃, M.W 230.26), r,s-ibuprofen (C₁₃H₁₈O₂, M.W 206.28), dexibuprofen (S-Ibuprofen, C₁₃H₁₈O₂, M.W 206.28), indomethacin (C₁₉H₁₆ClNO₄, M.W 357.79), acetaminophen (M.W 151.17), mefenamic acid (C₁₅H₁₅NO₂, M.W 241.29), chlorocinnazine 2HCl (C₂₆H₂₇N₂Cl. 2HCl, MW: 475.88), loxoprofen (C₁₅H₁₈O₃, MW: 246.31), fenoprofen (C₁₅H₁₄O₃, MW: 242.27), ketoprofen (C₁₆H₁₄O₃, MW: 254.29), pranoprofen (C₁₅H₁₃NO₃, MW: 255.27), meclofenamic acid (C₁₄H₁₁Cl₂NO₂, MW: 296.15) and salts thereof, sulindac (C₂₀H₁₇FO₃S, MW: 356.42), piroxicam (C₁₅H₁₃N₃O₄S, MW: 331.35), meloxicam (C₁₄H₁₃N₃O₄S₂, MW: 351.41), tenoxicam (C₁₃H₁₁N₃O₄S₂, MW: 337.38), diclofenac (C₁₄H₁₁Cl₂NO₂, MW: 296.15), aceclofenac (C₁₆H₁₃Cl₂NO₄, MW: 354.19), rebamipide (C₁₉H₁₅ClN₂O₄, MW: 370.79), enalapril maleate (C₂₀H₂₈N₂O₅, MW: 492.52), captopril (C₉H₁₅NO₃S, MW: 217.29), ramipril (C₂₃H₃₂N₂O₅ MW: 416.52), fosinopril (C₃₀H₄₆NO₇P, MW: 563.67), benazepril (C₂₄H₂₈N₂O₅, MW: 424.50), quinapril (C₂₅H₃₀N₂O₅, MW: 474.99) hydrochloride, temocapril (C₂₃H₂₈N₂O₅S₂ MW: 476.62), cilazapril (C₂₂H₃₁N₃O₅ MW: 417.51), lisinopril (C₂₁H₃₁N₃O₅, MW: 405.50), valsartan (C₂₄H₂₉N₅O₃, MW: 435.53), losartan potassium (C₂₂H₂₂ClKN₆O MW: 461.01), irbesartan (C₂₅H₂₈N₆O MW: 428.54), cetirizine hydrochloride (C₂₁H₂₅ClN₂O₃, MW: 388.90), diphenhydramine hydrochloride (C₁₇H₂₁NO. HCl, MW: 291.82), fexofenadine (C₃₂H₃₉NO₄, MW: 501.67), pseudoephedrine hydrochloride (C₁₀H₁₅NO HCl, MW: 201.70), methylephedrine hydrorchloride (C₁₁H₁₇NO.HCl, MW: 215.72), dextromethorphan hydrobromide (C₁₈H₂₅NO HBr H₂O, MW: 370.33), guaifenesin (C₁₀H₁₄O₄, MW: 198.22), noscapine (C₂₂H₂₃NO₇, MW: 413.43), tri-metoquinol hydrocloride (C₁₉H₂₃NO₅. HCl, MW: 399.87), doxylamine succinate (C₁₇H₂₂N₂O, C₄H₆O₄, MW: 388.5), ambroxol (C₁₃H₁₈Br₂N₂O, MW: 378.11), letosteine (C₁₀H₁₇NO₄S₂, MW: 279.37), sobrerol (C₁₀H₁₈O₂, MW: 170.25), bromhexine hydrochloride (C₁₄H₂₀Br₂N₂HCl, MW: 412.59), chlorpheniramine maleate (C₁₆H₁₉ClN₂. C₄H₄O₄, MW: 390.87) and optical isomers thereof, but are not limited thereto.

The foregoing acidic drugs are contained in an amount of 0.1 to 70% by weight, preferably 10 to 55% by weight, based on the total weight of the capsule filling material.

Hereinbelow, the present invention will be explained in detail, primarily referring to Naproxen that has the lowest dissolution rate among the previously listed acidic drugs. However, it will be apparent to those skilled in the art that the present invention is not limited thereto but can be applied to any of the hardly soluble acidic drugs.

The solvent system according to the present invention comprises a cation acceptor as a component. The term “cation acceptor” used herein refers to anion species which can take an cation upon dissociation into an anion and a cation, Bronsted base and Lewis base which can take hydrogen ion, and its examples include any one selected from the group consisting of pharmaceutically acceptable basic compounds (for example, KOH, NaOH), metallic salts of weak acids (for example, sodium acetate, potassium acetate, potassium citrate, sodium citrate), amines (for example, prolamine, di-ethanolamine, mono-ethanolamine, tri-ethanolamine, methylglucamine), or amino acids (for example, lysine, threonine, cystein) and a mixture of one or more thereof, but are not limited thereto. These cation acceptors may increase the solubility of the acidic drugs by readily taking hydrogen ion in the carboxyl group of the acidic drug.

Among the cation acceptors, hydroxide species that react with the acidic drug include sodium hydroxide (NaOH), potassium hydroxide (KOH), magnesium hydroxide (Mg(OH)₂), calcium hydroxide (Ca(OH)₂) and the like, with potassium hydroxide being the most preferred. Potassium of the potassium hydroxide has an atomic number greater than sodium. In the same element group, as the atomic number is bigger, the ionization tendency is increased. This is because the distance between a nucleus and an electron in the outermost shell is far and the force of the nucleus pulling the electrons is weak, whereby the ionization can readily occur to form a bond with a negatively charged ion. For these reasons, the potassium hydroxide can advantageously be used in the preparation of a salt of the acidic drug in the ionized state.

The basic compounds such as KOH and NaOH are used in an amount to make the hydroxyl ion (—OH) content of 0.2 to 1 mole per mole of the acidic group of the hardly soluble acidic drug. Especially, the hydroxide species are more preferably used in the same amount with water. If the hydroxide species are used in an excessive amount, the disintegration delay may occur due to the increase of pH.

Among the foregoing cation acceptors, the metallic salts of weak acids are preferably used in an amount of 0.1 to 2 mole per mole of the acidic group of a hardly soluble acidic drug. If the amount exceeds the foregoing range, the disintegration delay may occur due to the increase of pH.

Among the foregoing cation acceptors, the amines are used in an amount of 0.1 to 2 moles per mole of the acidic group of a hardly soluble acidic drug. Since the amines have abundant electrons in themselves, they can readily take cations. Accordingly, they can increase the ionization tendency of the acidic drug, thereby increasing solubility. If the amines are used in amount of over 2 moles with respect to the acidic drug, there is a problem of capsule stability associated with disintegration or dissolution, which makes it improper.

The mixed use of the foregoing cation acceptors may result in more preferred results and this feature forms another preferred aspect of the present invention. When the foregoing cation acceptors are used in combination, the total amount of the mixed cation acceptors is used in the range of 0.1 to 2 moles per mole of the acidic group of a hardly soluble acidic drug. Upon the mixed use, the hydroxide species may be more preferably used in the same amount with water and other cation acceptors can be used regardless of the amount of water and the hydroxide species.

It is more preferable that the amount of water needed in the solvent system of the present invention is 50% or more for the cation acceptor.

In the solvent system of the present invention, the surfactant serves as a co-solvent or a dissolution aid to promote drug dissolution and mainly comprises materials with the hydrophilic and hydrophobic properties. In particular, the surfactants for use in the present invention have a HLB (Hydrophilic Lipophilic Balance) value of 3 to 40, preferably 5 to 30 and can be used alone or in combination of two or more. Preferred examples of such surfactants are as follows:

i) Reaction products of natural or hydrogenated vegetable oils and ethylene glycol; that is, polyoxyethylene glycolated natural or hydrogenated vegetable oils; for example, polyoxyethylene glycolated natural or hydrogenated castor oils, such as the products commercially available under the trade name of CREMOPHOR RH 40, CREMOPHOR RH 60, CREMOPHOR EL, NIKKOL HCO-40, NIKKOL HCO-60, etc., with CREMOPHOR RH 40 and CREMOPHOR EL being particularly preferred.

ii) Polyoxyethylene sorbitan fatty acid esters; for example, mono- and tri-lauryl, palmityl, stearyl and oleyl esters of polyoxyethylene sorbitan fatty acids, such as products commercially available under the trade name of TWEEN, which includes Tween 20, 21, 40, 61, 65, 80, 81, 85, 120, with Tween 20, Tween 60 and Tween 80 being preferred.

iii) Transesterification products of natural vegetable oil tri-glyceride and polyalkylene polyol; for example, commercially available surfactants such as Labrafil M 2125 CS, Labrafil M 1944 CS, or Labrafac CC, Labrafac PG; and Labrasol.

iv) Polyoxyethylene fatty acid esters, for example, polyoxyethylene(8) stearate (trade name: MYRJ 45), polyoxyethylene(30) mono-laurate (trade name: TAGAT L), polyoxyethylene(20) stearate (trade name: MARLOSOL 1820), polyoxyethylene(15) oleate (trade name: MARLOSOL OL 15), trade name: CETIOL HE; polyoxyethylene stearic acid esters, for example, polyoxyethylene-polyoxypropylene copolymers, such as products of the trade name of PLURONIC and EMKALYX; polyoxyethylene-polyoxypropylene block copolymers, for example, products commercially available under the trade name POLOXAMER, specifically POLOXAMER 188, 124, 237, 338, 407, mono-, di- and mono-/di-glyceride, particularly, esterification products of caprylic acid or capric acid and glycerol, surfactants mainly comprising caprylic acid/capric acid mono- and di-glyceride, for example, Imbitor.

v) Sorbitan fatty acid esters; for example, sorbitan mono-laurate, sorbitan mono-palmitate, sorbitan mono-stearate, sorbitan tri-stearate, sorbitan mono-oleate, sorbitan tri-oleate, etc., such as products commercially available under the trade name of Span; polyethylene glycol fatty acid esters, which are classified to stearates, laurates, oleates according to the bonded fatty acid, with polyethylene glycol mono-oleate being preferred, for example, trade name of MYO-2, MYO-6, MYO-10 etc.

vi) Propylene glycol mono- and di-fatty acid ester, for example, propylene glycol dicaprylate, such as trade name of MIGLYOL 840; propylene glycol dilaurate, propylene glycol hydroxystearate, propylene glycol iso-stearate, propylene glycol laurate, propylene glycol lysine oleate, propylene glycol stearate, etc., for example, trade name of SEFSOL 218 and CAPRYOL 90, CAPRYOL PGMC, LAURO GLYCOL FCC or LAURO GLYCOL 90; MAISINE 35-1 (glyceryl mono-linolate), PECEOL (glyceryl mono-oleate), GELUCIRE 44/14 (lauroyl polyoxyl-32 glyceride) and GELUCIRE 33/01 (fatty acid glycerol ester),

vii) Pharmaceutically acceptable C₁₋₅ alkyl or tetrahydrofurfuryl di- or partial-ether of low molecular mono- or poly-oxy-alkanediol, for example, diethylene glycol monoethyl ether, commercially available under the trade name TRANSCUTOL;

viii) Polyoxyethylene fatty acid ethers, for example, polyoxyethylene (10) oleyl ether (trade name: BRIJ 96), polyoxyethylene (15) oleyl ether (trade name: VOLPO 015), polyoxyethylene (30) oleine ether (trade name: MARLOWET OA30), polyoxyethylene (20) C₁₂-C₁₄ fatty acid ether).

ix) Polyoxyethylene-polyoxypropylene copolymer, for example, trade name SYPORONIC PE L44, SYPORONIC F127.

Among them, the surfactant is preferably selected from the group consisting of CREMOPHOR RH40 (Polyoxyl 40 hydrogenated castor oil), CREMOPHOR EL (Polyoxyl 35 castor oil), LABRASOL (polyethylene glycol caprylate/caprate), TRANSCUTOL (diethylene glycolmono-ethyl ether), TWEEN (polysorbate) 20, 21, 40, 61, 65, 80, 81, 85, 120, POLOXAMER 124, 188, 237, 338, 407 (polyoxyethylene-polyoxypropylene), NIKKOL HCO-40 (polyoxyethylene glycolated natural or hydrogenated castor oil), MYRJ 45 (polyoxyethylene(8)stearate), TAGAT L (polyoxyethylene (30) mono-laurate), MARLOSOL 1820 (polyoxyethylene(20) stearate), MARLOSOL OL 15 (polyoxyethylene(15) oleate), BRJJ 96 (polyoxyethylene(10) oleyl ether), VOLPO 015 (polyoxyethylene(15) oleyl ether), MARLOWET OA30 (polyoxyethylene(30) oley ether), MARLOWET LMA 20 (polyoxyethylene(20) oleyl ether), SYPERONIC PE L44 (polyoxyethylene-polyoxypropylene copolymer), SYPERONIC F127 (polyoxyethylene-polyoxypropylene copolymer, LABRAFIL M 2125 CS (linoleoyl macrogol glycerides), LABRAFC PG (propylene glycol dicaprylocaprate), Imbitor (caprylic acid/capric acid mono- and di-glyceride), sorbitan mono-stearate, sorbitan tri-stearate, sorbitan mono-oleate, polyethylene glycol mono-oleate, MIGLYOL 840 (propylene glycol dicaprylate), GELUCIR 44/14 (lauroyl polyoxyl-32 glyceride) and the mixtures thereof.

It is more preferable that the surfactant is selected from the group consisting of CREMOPHOR RH40 (Polyoxyl 40 hydrogenated castor oil), CREMOPHOR EL (Polyoxyl 35 castor oil), LABRASOL (polyethylene glycol caprylate/caprate) and TRANSCUTOL (diethylene glycol mono-ethyl ether). The Polyoxyl 40 hydrogenated castor oil (CREMOPHOR RH 40) may be the most preferable. CREMOPHOR RH 40 which is a derivative of castor oil is obtained by the synthesis and purification process. It has a solidifying point of 20 to 28 C°, a saponification value of 50 to 60, hydroxy value of 60 to 70 and pH of 6 to 7 in 10% solution. It is light white or yellow and has a HLB value HLB of 14 to 16. It is soluble in water, ethanol, 2-propanol, n-propanol, ethyl acetate, chloroform, toluene, Xylene, etc.

The above-described surfactants can be used alone or as a mixture of two or more components and can be properly selected according to the properties of the solvent system. It can be used in the amount of 0.1 to 50%, preferably 0.2 to 40%, most preferably 0.2 to 30%, based on the weight of the solvent system.

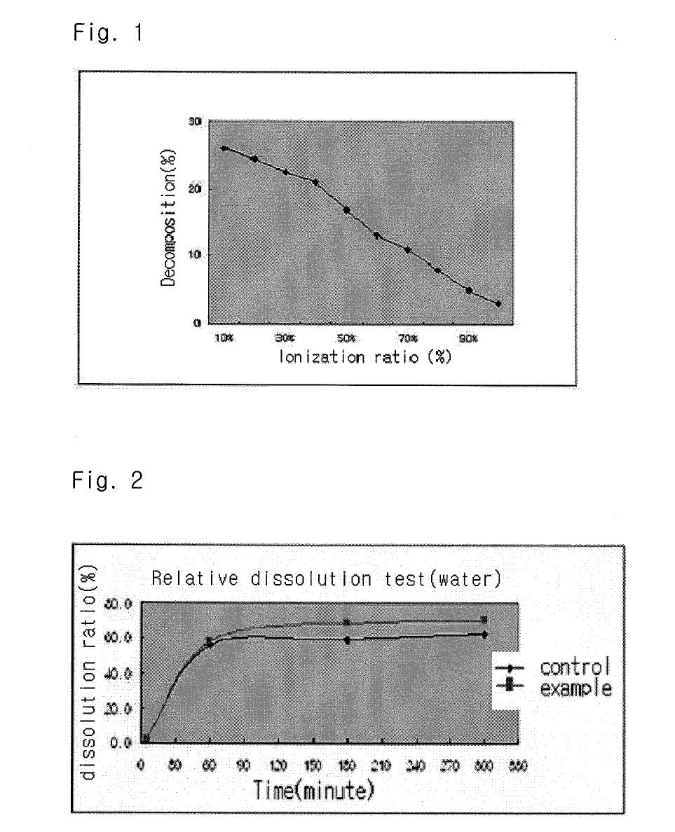

According to the present invention, the improvement of the dissolution rate is accomplished by selecting a vehicle capable of dissolving both hydrophilic water and a hydrophobic drug. The ionization degree of the acidic drug can have an effect on stabilization of the drug. In an accelerated state or acidic condition, the carboxylic acid of the acidic drug and the alcohol group (—OH) of polyethylene glycol undergo esterification reaction, which exerts a great influence to the stabilization of the drug. An experiment example showing such a circumstance is shown in FIG. 1. which indicates that the esterification is significantly reduced when the drug is ionized.

Also, the water content in the filling material may have an effect on the esterification reaction. If a large amount of water is contained in a capsule, water in the filling material can be migrated to the shell, whereby the dried capsule appearance can be changed and the drying time is extended. In general, the migration of water in the filling material does not occur in all capsules. It is known that about 10% of water can exist in a system containing a hydrophilic component. Table 1 shows the experimental data showing the degree of such migration. That is, Table 1 shows the ionization of 10% acidic drug according to the PEG 400 and water contents. According to the experimental result, it was noted that the filling material of the capsule containing water of 10% or less can optimally reduce the esterification of the acidic drug.

TABLE 1

| | | Dissociation of |
|---|---|---|
| Ionization of | Water | agent after 7 days |
| drug (%) | (%) | at 105° C. (%) |
| 50 | 5.0 | 14.9 |
| 50 | 10.0 | 8.8 |
| 30 | 1.4 | 24.3 |
| 30 | 5.0 | 19.3 |
| 0 | 0.0 | 29.8 |
| 0 | 5.0 | 24.6 |

Explaining the chemical mechanism of the acidic drug according to the present invention in detail, the esterification mainly occurs in the acidic drug, i.e. the derivative of carboxylic acid, in which the esterification may form a more stable resonance in the filling material state if there is a electron donating groups to a substrate of the carboxyl group of the drug.

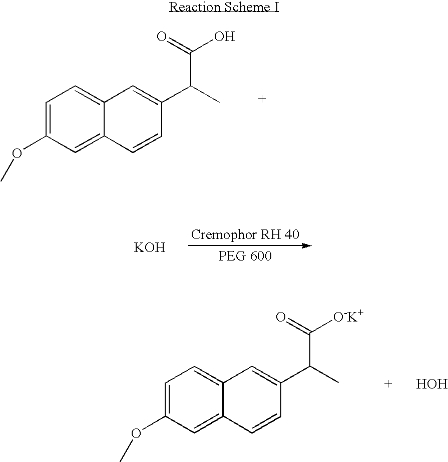

As can be explained in the above Reaction Scheme (I), the acidic hydrogen of carboxylic acid can maintain a condition suitable for dissociation and, thus, can readily be ionized. This is because oxygen of the carboxyl group of carboxylic acid and oxygen of the hydroxyl group can form a resonance structure. This theory can be applied to a reaction performed in a solvated state and, in practice, the actual stabilized state of a main reaction mechanism of acidic drug such as Naproxen is accomplished by the following Reaction Scheme (II).

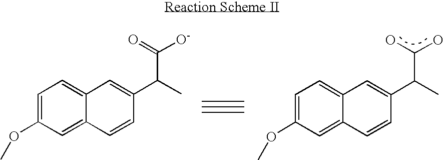

The features of the acidic drug in the ionized state thus obtained are associated with conjugate acid-conjugate base and thus are reduced to its original state when mixed with water.

Another example that has an effect on the resonance of carboxylic acid in the acidic drug can be found in substrate having electron-donor properties around carboxylic acid, the representative example of which includes benzene derivatives, alkyl groups and methyl groups having a double or triple bond. However, though all the prepared filling material maintain their salt states, the drugs may be reduced to their original state to form crystals, in view of the drug release aspect, whereby they are not exist in the salt states, causing deterioration in effects of the drugs. In order to solve these problems, in the present invention, a surfactant which is the most effective in functioning as both a hydrophobic part and a hydrophilic part, as described above is selectively used.

The solvent system according to the present invention comprises polyethylene glycol, a liquid filler as a basic component, which has preferably an average molecular weight of about 200 to 800, more preferably an average molecular weight of 600.

Other basic components which can be used in the present invention include, but not limited thereto, analogues of polyethylene glycol, such as tetra glycol, polyethylene glycol ethers of various alcohols, that is, polyethylene glycol ether of tetrahydroperfuryl-alcohol, and polyethylene glycol copolymers.

According to the present invention, the polyethylene glycol is selected as a component to minimize the esterification (RCOOR′) of carboxylic acid (RCOOH) of the acidic drug and hydroxyl group (R′OH) of polyethylene glycol.

In general, the optimal conditions for chemical reactions are diverse, including temperature, pressure, catalyst, mole concentration, viscosity, etc. The PEG was selected as a component to construct the filling material of the drug with minimized influence on the solvent system according to the present invention. The effect of the selected polyethylene glycol is not limited thereto but also include a more important function. When the drug has a low molecular weight, the polyethylene glycol having a high molecular weight has a role to inhibits the increase of drug migration to the shell as time goes on and reduces the migration rate.

The pharmaceutical preparation according to the present invention may further comprise propylene glycol, glycerin, polyvinyl pyrrolidone, propyl carbonate, anti-oxidants, low-molecular weight alcohols such as ethanol, which are commonly used as a pharmaceutical vehicle.

The optimal conditions to maintain chemical stability of the acidic drug include a high ionization degree of the drug, a small amount (as small as possible) of glycerin, ethanol, propylene glycol, propylene carbonate as a vehicle contained in the filling material, and use of a component having a small amount of —OH group. The polyethylene glycol having a large molecular weight is preferably used alone or in combination with a polyethylene glycol having a small molecular weight. Also, water is contained in a maximum amount as long as the drug migration to the shell is inhibited and potassium hydroxide rather than sodium hydroxide is preferably used to maximize the solubility of the drug. Therefore, the present invention is characterized by the foundation of an optimal ratio of the various components to maximize bioavailability of a drug in a solvent system, a pharmaceutical preparation comprising the solvent and a formulated capsule comprising the preparation.

Also, according to the present invention, it is possible to provide a soft capsule with improved disintegration rate by dissolving a hardly soluble acidic drug using the solvent system and by using a specific plasticizer composition in the capsule shell. That is, since glycerin which has been conventionally used as a component of the shell is not contained, it is possible to inhibit the esterification reaction caused by the glycerin, thereby complementing the defects associated with delay of disintegration. In the encapsulation of the filling material using the solvent system according to the present invention, it is preferable to use a shell composition comprising 30 to 45% by weight of gelatin, 15 to 24% by weight of Esitol and sorbitans, and 25 to 34% by weight of water.

Therefore, in another aspect of the present invention, there is provided a soft capsule comprising a shell composition comprising 30 to 65% of gelatin, 10 to 40% of Esitol and a sorbitan, 1 to 15% of water based on the weight of the solution of the pharmaceutical preparation, as described above and the dried shell, and as needed, a preservative, a coloring agent, flavoring agent, a fragrance, a light blocking agent and a disintegration enhancer. By selecting such pharmaceutical formulation, it is possible to reduce the reactivity between the drug and components of the shell. Also, by this system, it is possible to obtain the content uniformity by minimization of migration of the filling material in the soft capsule to the shell and to minimize the esterification which is a main cause for content reduction, since the shell does not contain glycerin. The following requirements are for production of gelatin capsules under optimal process conditions.

Gelatin: 190 to 210 Bloom

Temperature of gelatin mass: 58 to 62° C.

Drying temperature: 22 to 24° C.

Drying humidity: 22 to 24% RH (30% RH or less)

### EXAMPLE

Now, the present invention will be explained in detail through the following Examples. However, the Examples are not for limitation of the present invention.

### Example 1

### Comparison of Solubility in Various Vehicles

Dexibuprofen and Naproxen which are representative hardly soluble drugs were measured for solubility using the following vehicles and the results are shown in Table 2 below.

TABLE 2

| | Dexibuprofen | Naproxen |
|---|---|---|
| PEG 400 | 145% | 8.1% |
| Tween 80 | 95% | 15.1% |
| Capryol 90 | 98% | 5.1% |
| Labrafil M 2125 CS | 45% | X |
| Labrasol | 88% | 19.2% |
| Labrafac CC | 44% | X |
| Transcutol P | 180% | 26.7% |
| Cremophor RH 40 | 86% | 18.2% |

The above solubility test for the two drugs were conducted under the same condition (at room temperature). It was shown that Naproxen had a significantly low solubility or was insoluble in the all tested surfactant (X represents “being insoluble”).

### Example 2

### Solubility Test of Naproxen

The solubility of Naproxen was examined using the surfactants described in Table 3 as a subsidiary component (vehicle) for the filling material.

TABLE 3

| | Solubility | | Solubility |
|---|---|---|---|
| Surfactant | (%) | Surfactant | (%) |
| Labrasol | 19.2 | Lauro glycol FCC | 2.5 |
| Cremophor RH | 18.2 | Lauro glycol 90 | 3.3 |
| 40 | | | |
| Tween 80 | 15 | Transcutol P | 26.7 |
| Capryol 90 | 5 | Peceol | 4.2 |
| Capryol PGMC | 5 | Labrafac PG | 1.7 |
| Labrafil M | X | Labrafac CC | X |
| 2125 CC | | | |
| Labrafil M | X | Tri-acetin | 5 |
| 1944 CS | | | |

As can be seen from the result of Table 3, it was shown that surfactants with excellent solubility, particularly having an HLB (Hydrophilic Lipophilic Balance) value of 5 to 16 are suitable for the solvent system according to the present invention. Also, it was noted that even when the vehicles, i.e. the surfactants were used alone or as a combination, drug release was improved.

### Example 3

On the basis of the result of the solubility test in Example 2, pharmaceutical formulations described in Table 4a and Table 4b below were prepared and examined for their properties according to the methods described below. The content of each component was expressed in mg.

TABLE 4a

| | Formulation | | | | | | | | | | |
|---|---|---|---|---|---|---|---|---|---|---|---|
| | 1 | 2 | 3 | 4 | 5 | 6 | 7 | 8 | 9 | 10 | 11 |
| Naproxen | 250 | 250 | 250 | 250 | 250 | 250 | 250 | 250 | 250 | 250 | 250 |
| PEG 400 | | | 354.5 | 106.3 | | | | | | | |
| PEG 600 | 260 | 260 | | 247.6 | 330.2 | 330.2 | 330.2 | 439.5 | 240.0 | 449.9 | 360 |
| KOH | 30.6 | 35.0 | 30.6 | 34.8 | 34.8 | 34.8 | 34.8 | 35.1 | 35.05 | 35.05 | 35.05 |
| R.O. water | 61.3 | 35.0 | 61.3 | 61.3 | 61.3 | 61.3 | 61.3 | 35.5 | 35.05 | 35.05 | 35.05 |
| Transcutol P | | | | | | 23.7 | | | | | |
| Glycerin | 17.4 | 10 | | | | | | | | | |
| Labrafac cc | | | | | | | 23.7 | 20.0 | | | |
| Labrafac PG | | | | | | | | 20.0 | | | |
| Tween 80 | | | | | 23.7 | | | | 240 | 30 | 120 |
| Total | 619.3 | 590 | 696.4 | 700 | 700 | 700 | 700 | 800.1 | 800.1 | 800 | 800.1 |

TABLE 4b

| | Formulation | | | | | | | | | | |
|---|---|---|---|---|---|---|---|---|---|---|---|
| | 12 | 13 | 14 | 15 | 16 | 17 | 18 | 19 | 20 | 21 | 22 |
| Naproxen | 250 | 250 | 250 | 250 | 250 | 250 | 250 | 250 | 250 | 250 | 250 |
| PEG 600 | 420 | 382.9 | 369.9 | 369.9 | 360 | 360 | 390 | 360 | 454.3 | | |
| Cremophor RH | | | | 50 | | | | | | | |
| 40 | | | | | | | | | | | |
| KOH | 35.05 | 35.05 | 35.05 | 35.05 | 35.05 | 35.05 | 35.05 | 35.05 | 35 | | |
| R.O Water | 35.05 | 35.05 | 35.05 | 35.05 | 35.05 | 35.05 | 35.05 | 35.05 | 45 | | |
| Transcutol P | | | | | | | | | | 103 | 81 |
| Glycerin | | | | | | | | | 10 | | |
| Povidon | | | | | | | | | 5 | | |
| Tween 80 | 60 | 97.0 | 110 | 60 | 105 | 90 | 60 | 40 | | | |
| PG | | | | | | 10 | 30 | 40 | | | |
| Labrasol | | | | | | | | | | 310 | 248 |
| Maisine 35-1 | | | | | | | | | | 69 | 55 |
| Labrafil M | | | | | | | | | | 69 | 55 |
| 1944 CS | | | | | | | | | | | |
| Lauro glycol | | | | | | | | | | 138 | 110 |
| 90 | | | | | | | | | | | |
| SPAN 80 | | | | | | | | 40 | | | |
| Linoleic | | | | | 20.0 | 20.0 | | | | | |
| acid | | | | | | | | | | | |
| Total | 800.1 | 800 | 800 | 800 | 805.1 | 800.1 | 770.1 | 800.1 | 799.3 | 939 | 799 |

As can be seen from the results of the following experiments, though not all the formulations listed in the above Table 4a and Table 4b showed satisfactory results, it was sure that the formulations using the surfactants according to the present invention showed somewhat improved results, as compared to the conventional formulations. Therefore, it was noted that the dissolution rate could be improved to accomplish the objects of the present invention by selecting a vehicle which can dissolve both hydrophilic water and a hydrophobic drug.

### Example 4

### Preparation of High Concentration Solution of Acidic Drug

Following the compositions described in Tables 5a through Table 5e, the acidic drugs was mixed with polyethylene glycol to form a thoroughly wet mixture and a hydroxide solution was slowly added thereto. The mixture was confirmed to turn to be a completely clear solution, followed by deaeration. The phenomenon that the wet mixture of the active drug and polyethylene glycol became a clear solution upon addition of the hydroxide solution is interpreted to that hydrogen of the hydroxyl group of the carboxylic acid in the acidic drug was released and formed a salt together with an alkali metal element of the hydroxide, that is, the drug had been ionized. This equilibrium can be maintained while the drug is in the filling material. However, when the drug contacts water, it returns to its carboxylic acid form for stabilization. Thus, the prepared solution of the drug maintains the clear solution state until the capsule is opened in water upon disintegration test and then, the drug is reduced in water from its salt state.

The compositions of the following Examples were representatively established to make prescriptions which can be dissolved, on the basis of solubilities of Naproxen and Dexibuprofen. However, it is apparent to those skilled in the art that other various compositions and components can be selected within the scope of the present invention, considering the description presented herein. The components are expressed in mg.

TABLE 5a

| | Example No. | | | | | | |
|---|---|---|---|---|---|---|---|
| | 4-1 | 4-2 | 4-3 | 4-4 | 4-5 | 4-6 | 4-7 |
| Naproxen | 250 | 250 | 250 | 250 | 250 | 250 | 250 |
| PEG 600 | 240 | 360 | 382.9 | 360 | 310 | | |
| Potassium | 35.05 | 35.05 | 35.05 | 35 | 35.05 | 30 | 35 |
| hydroxide | | | | | | | |
| Water | 35.05 | 35.05 | 35.05 | 35 | 35.05 | 30 | 35 |
| Polyoxy- | | | | 30 | | | |
| ethylene | | | | | | | |
| mono-oleate | | | | | | | |
| Propylene | | | | | | 20 | |
| glycol | | | | | | | |
| Tetra glycol | | | | | | | 400 |
| Tween 80 | 240 | 120 | 97.0 | 85 | | | |
| Cremophor | | | | | 155 | 120 | 105 |
| RH40 | | | | | | | |

TABLE 5b

| | Example No. | | | | | | |
|---|---|---|---|---|---|---|---|
| | 4-8 | 4-9 | 4-10 | 4-11 | 4-12 | 4-13 | 4-14 |
| Naproxen | 250 | 250 | 250 | 250 | | | |
| Dexibuprofen | | | | | 300 | 300 | 300 |
| PEG 600 | 400 | 150 | 320 | 440 | 330 | 330 | 330 |
| Potassium | 30 | 20 | 30 | 35 | 30 | 30 | 35 |
| hydroxide | | | | | | | |
| Water | 30 | 20 | 30 | 35 | 30 | 30 | 35 |
| Polyethylene | | 250 | | | | | |
| glycol | | | | | | | |
| Propyl | | 30 | | | | | |
| carbonate | | | | | | | |
| Propylene | 30 | | | | | | |
| glycol | | | | | | | |
| Cremophor RH40 | 200 | 250 | 200 | | 300 | | |
| Transcutol P | | | | 40 | | | |
| Tween 80 | | | | | | 30 | |
| Labrasol | | | | | | | 300 |

TABLE 5c

| | Example No. | | | | | |
|---|---|---|---|---|---|---|
| | 4-15 | 4-16 | 4-17 | 4-18 | 4-19 | 4-20 |
| Dexibuprofen | 300 | 300 | 300 | 300 | 300 | 300 |
| PEG 600 | 350 | 350 | 350 | 400 | 400 | 350 |
| Potassium | 35 | 25 | 25 | 20 | 20 | 20 |
| hydroxide | | | | | | |
| Water | 35 | 25 | 25 | 20 | 20 | 20 |
| Propyl | | | | | | 50 |
| carbonate | | | | | | |
| Mono-ethanol | | | | | | 50 |
| amine | | | | | | |
| Nikkol HCO 40 | 300 | | | | | |
| Poloxamer 188 | | 10 | | | | |
| Myrj 45 | | | 30 | | | |
| Syperonic PE144 | | | | 24 | | |
| Capryol 90 | | | | | 15 | |
| Marlowet OA 30 | | | | | | 15 |

TABLE 5d

| | Example No. | | | | | |
|---|---|---|---|---|---|---|
| | 4-21 | 4-22 | 4-23 | 4-24 | 4-25 | 4-26 |
| Dexibuprofen | 300 | 300 | 300 | 300 | 300 | 300 |
| PEG 600 | 400 | 350 | 350 | 400 | 250 | 300 |
| Potassium | 20 | 20 | 20 | 20 | 20 | 20 |
| hydroxide | | | | | | |
| Water | 20 | 20 | 20 | 20 | 20 | 20 |
| Glycerin | | 50 | | | | |
| Propylene | | | 50 | | | |
| glycol | | | | | | |
| Potassium | | 50 | | | | |
| citrate | | | | | | |
| Sodium citrate | | | 50 | 50 | | |
| Potassium | | | | | 50 | |
| acetate | | | | | | |
| Diethanol amine | | | | | | 50 |
| Tri-ethanol | 50 | | | | | |
| amine | | | | | | |
| Nikkol HCO 40 | | | | 300 | | |
| Labrasol | | | | | 300 | |
| Tween 80 | | | | | | 30 |
| Poloxamer 188 | | | 10 | | | |
| Myrj 45 | | 30 | | | | |
| Syperonic PE144 | 24 | | | | | |

TABLE 5e

| | Example No. | | | | | |
|---|---|---|---|---|---|---|
| | 4-27 | 4-28 | 4-29 | 4-30 | 4-31 | 4-32 |
| Dexibuprofen | 300 | 300 | 300 | 300 | 300 | 300 |
| PEG 600 | 330 | 250 | 300 | 300 | 300 | 300 |
| Potassium | 20 | 20 | 20 | 10 | 40 | 10 |
| hydroxide | | | | | | |
| Water | 20 | 20 | 20 | 10 | 40 | 10 |
| Potassium | | 20 | 20 | | | |
| citrate | | | | | | |
| Povidon | | | 20 | | | |
| L-lysine | | | | | 100 | |
| Methylglucamine | | | | | | 100 |
| Diethanol amine | 50 | 50 | | | | |
| Tri-ethanol | | | | 100 | | |
| amine | | | | | | |
| Cremophor RH40 | 20 | 20 | | | | |
| Tween 80 | | | | 5 | 3.5 | 5 |

### Example 5

### Dissolution Rate Test 1

A soft capsule comprising the filling material prescribed according to the present invention and a tablet as a control for comparison were examined for the dissolution rate. As the filling material prepared according to the present invention, the formulation of Example 4-11 was used, the capsule shell was formed using a composition comprising 43.2% of gelatin, 24.8% of sorbitan and Esitol and 32% of water. The comparative formulation (control) was Naxen tablet (produced by ChongKunDang, Lot No. DA005), which is one of the commercially available according to the provision of the therapeutic equivalence in the Korea pharmacopoeia and the results of the test of the dissolution rate in water are shown in FIG. 2. As shown in FIG. 2, the prescription according to the present invention using an excipient medium as a vehicle showed improved dissolution rate up 12%, as compared to the tablet selected as control.

The improved dissolution rate was confirmed not only in water, but also under the condition according to the dissolution test described in the paragraph of Naproxen tablet in the Korea Pharmacopoeia (0.1 mol/L phosphate buffer (pH 7.4) 900 ml, the absorption is measured at 332 nm 45 minute later after the initiation of the dissolution test according to the second method, with over 80% being suitable). The results of the dissolution rate test in phosphate buffer are shown in Table 6 and the dissolution rate graph is shown in FIG. 3.

TABLE 6

| | | Test time (min) | | |
|---|---|---|---|---|
| | | 15 | 30 | 45 |
| The | Dissolution | 83.8 | 93.2 | 95.4 |
| present | rate | | | |
| invention | Standard | 1.46 | 2.73 | 1.10 |
| | deviation | | | |
| Control | Dissolution | 85.0 | 90.1 | 01.5 |
| | rate | | | |
| | Standard | 2.13 | 2.46 | 0.15 |
| | deviation | | | |

### Example 6

### Dissolution Rate Test 2

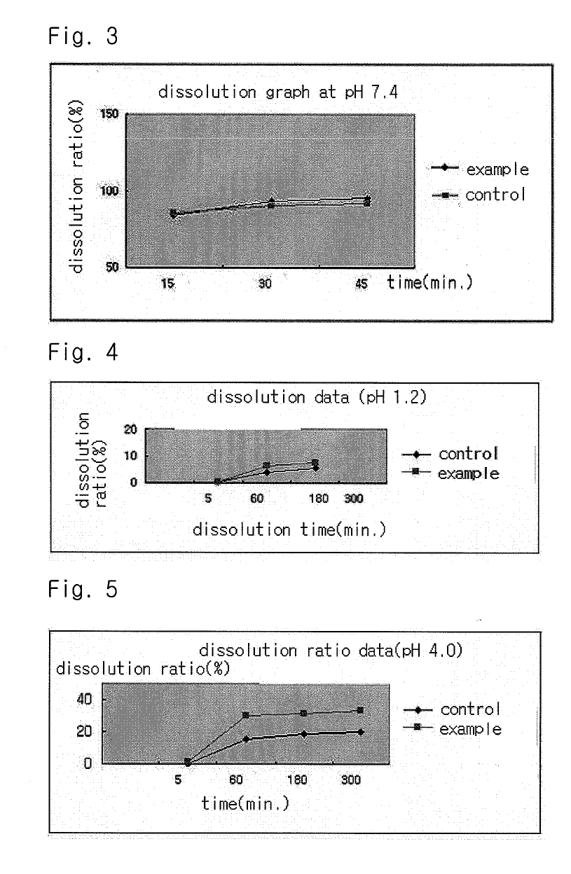

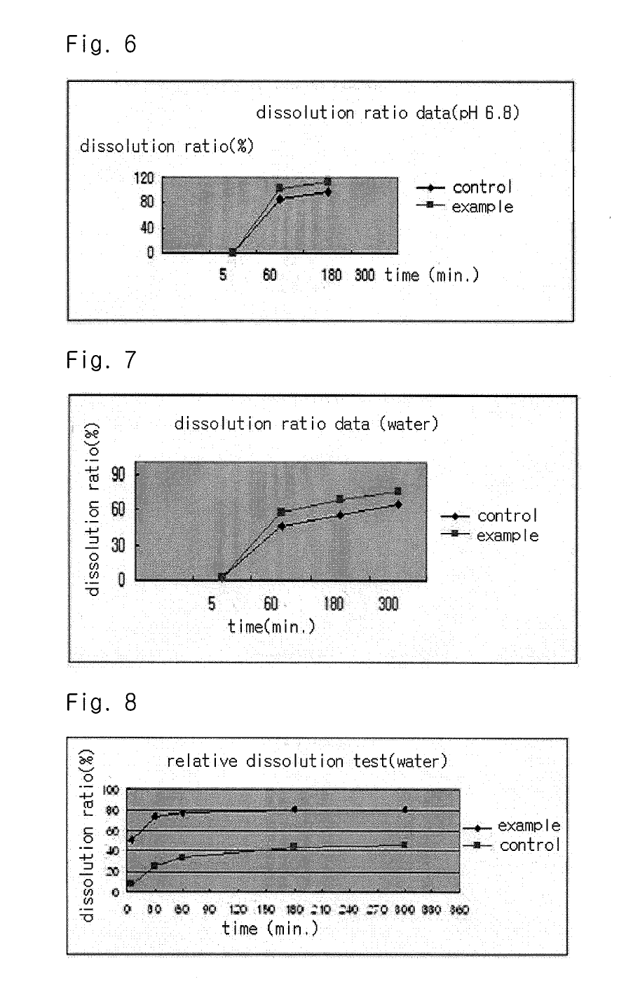

Following the method used in Example 5, the prescription (Example) according to the present invention and the prescription (Control) disclosed in Example IV of Korean Patent Publication No. 1994-0006270 were formulated into soft capsules and examined for the dissolution rate in solutions with different pH described in the index of the therapeutic equivalence test and the results are shown in Table 7 and FIGS. 4, 5, 6 and 7.

TABLE 7

Dissolution time (min)

| | 5 | 60 | 180 | 300 |
|---|---|---|---|---|
| pH 1.2 (control) | 0.4 | 3.8 | 5.4 | |
| pH 1.2 (example) | 0.5 | 5.4 | 7.5 | |
| pH 4.0 (control) | 0.2 | 15.3 | 18.9 | 19.8 |
| pH 4.0 (example) | 1.1 | 30.1 | 31.5 | 33.4 |
| pH 6.8 (control) | 0.3 | 85.0 | 98.0 | |
| pH 6.8 (example) | 0.2 | 101.2 | | |
| Water (control) | 2.4 | 46.3 | 55.8 | 65.2 |
| Water (example) | 2.3 | 57.7 | 68.6 | 75.2 |

As can be seen from the above results, the prescription according to the present invention showed improved dissolution rates, particularly by 38.9% (pH 1.2), 66.7% (pH 4.0), 16.5% (pH 6.8) and 22.9% (water) at 180 minutes later, as compared to the control.

### Example 7

### Dissolution Rate Test 3

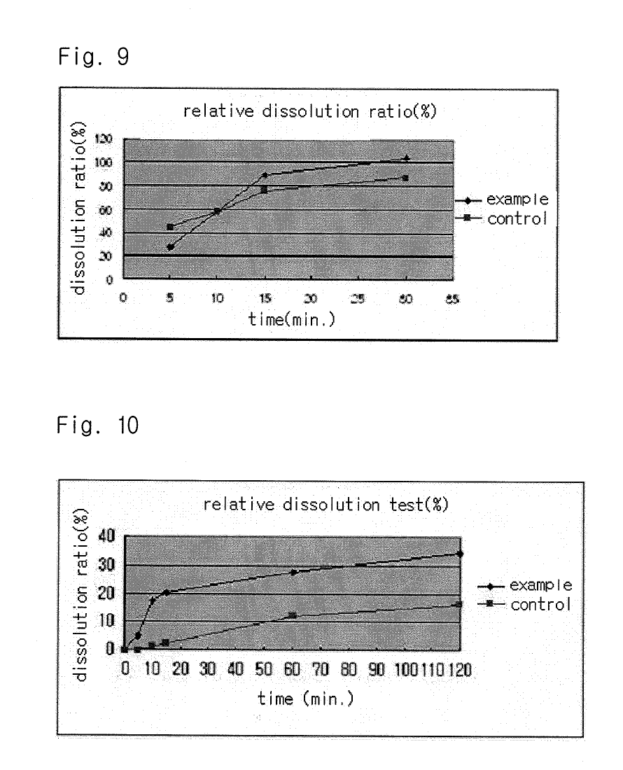

Following the method used in Example 5, the Dexibuprofen prescription of Example 4-13 according to the present invention and Daxpen tablet (Bi-nex, Lot No. 0203002), as a control according to the therapeutic equivalence index, were examined for the dissolution rate in water and at pH 6.8, and the results are shown in Table 8 and FIGS. 8 and 9.

TABLE 8

| | Dissolution time (min) | | | | |
|---|---|---|---|---|---|
| | 5 | 30 | 60 | 180 | 300 |
| Water (control) | 7.20 | 25.47 | 33.19 | 43.29 | 46.05 |
| Water (example) | 50.44 | 73.32 | 77.32 | 80.95 | 80.35 |
| PH 6.8 (control) | 44.62 | 87.64 | | | |
| PH 6.8 (example) | 26.74 | 104.41 | | | |

As can be seen from the results, the prescription according to the present invention showed the dissolution rate improved by 19% (pH 6.8) and 74% (water) in this dissolution rate test 3.

### Example 8

### Dissolution Rate Test 4

Following the method used in Example 5, the Dexibuprofen prescription of Example 4-30 according to the present invention and Daxpen tablet (Bi-nex, Lot No. 0203002), as a control according to the therapeutic equivalence index, were examined for the dissolution rate in water and at pH 1.2, and the results are shown in Table 9 and FIG. 10.

TABLE 9

| | Dissolution time (min) | | | | |
|---|---|---|---|---|---|
| | 5 | 10 | 15 | 60 | 120 |
| pH 1.2 (control) | 0 | 1.3 | 2.2 | 12.1 | 16.3 |
| pH 1.2 (example) | 5.2 | 17.5 | 20.1 | 27.1 | 34.7 |

As can be seen from the above results, the prescription according to the present invention showed improved dissolution rate by about 2.1 times.

### Example 9

### Disintegration Test

The formulations used in Example 5 were subjected to the disintegration test. The disintegration test was conducted according to the method described in the general test method of the Korean pharmacopoeia. The results are shown in Table 10.

TABLE 10

| | | | |
|---|---|---|---|
| Test | Disintegration tester | Cycle | 30 |
| apparatus | | | times/minute |

| | | | | | Final test time | |
|---|---|---|---|---|---|---|
| | | No. of | Amount | Temp. | (min) | |
| | Solution | specimen | (ml) | (° C.) | Control | Test agent |
| Test | Water | 12 | 800 | 37° C. | Less than | Less than |
| solution | | | | | 10 min. | 10 min. |

| | | | | | |
|---|---|---|---|---|---|
| Test | Comparative | Validation | Korea | Validation | None |
| method | disintegration | | pharmacopoeia | | |

3 lots of test agents were prepared. As a result, the test agents (example) and control passed the acid resistant screen within 10 minutes, without exceeding the test standard of 20 minutes. All the test agents satisfied the standards of the disintegration test for an accelerated period of 6 months.

### Example 10

### Content Test

The prescription according to Example 4-11 was encapsulated without glycerin in the capsule shell and subjected to an accelerated period of 6 months to examine the migration of the filling material to the shell. The results are shown in Table 11 below.

TABLE 11

| Storing | | | | | | |
|---|---|---|---|---|---|---|
| conditions | Test items | Test standard | Initial | 2 months | 4 months | 6 months |
| 40° C. | Morphology | Transparent | Proper | Proper | Proper | Proper |
| 75% RH | | rectangular | | | | |
| | | soft capsule | | | | |
| | | containing | | | | |
| | | light yellow | | | | |
| | | to light | | | | |
| | | orange | | | | |
| | Confirmation | KP | Proper | Proper | Proper | Proper |
| | Weight deviation | Notification | Proper | Proper | Proper | Proper |
| | Disintegration test | KP | Proper | Proper | Proper | Proper |
| | Content | Average | 103.9% | 102.6% | 101.7% | 100.1% |
| | | 90.0 to | 103.6% | 102.6% | 102.1% | 101.1% |
| | | 110.0% | 103.2% | 102.0% | 100.7% | 100.8% |
| | | | 104.8% | 103.4% | 102.3% | 98.5% |

As can be seen in the results of Table 11, since the migration of the filling material in the soft capsule to the shell was minimized, it is possible to accomplish the content uniformity. Also, by not using glycerin in the shell, it is possible to provide a product with the esterification minimized, which otherwise causes reduction in the content.

The most important utility of the improved solvent system according to the present invention is to increase the bioavailability of drugs to be dissolved therein. Thus, by the solvent system according to the present invention, it is possible to minimize the migration of the filling material in a soft capsule to the shell, thereby providing the content uniformity and to minimize the esterification reaction which may cause the content reduction by not using the glycerin. Accordingly, as the disintegration and dissolution rates of a hardly soluble drug are improved, the drug in a solution can be more rapidly and uniformly released and absorbed at an absorption site, thereby increasing the bioavailability. Also, by using surfactants with various beneficial properties alone or as a mixture, it is possible to minimize crystallization due to the dissociation of hydrophilic components and hardly soluble drugs and to prepare the capsule shell without glycerin. Further, even in case of a hardly soluble drug, it is possible to provide a highly concentrated solution of the drug with a volume (size) that is small enough to allow easy swallowing by reducing the volume of the filling material.

What is claimed is:

1. A pharmaceutical preparation comprising a hardly water-soluble acidic drug and a solvent system,
wherein the solvent system comprises a pharmaceutically acceptable cation acceptor, 10 to 90% by weight of polyethylene glycol, 0.1 to 15% by weight of water and 0.1 to 50% by weight of a surfactant having an HLB value of 3 to 40 to improve the dissolution rate of the drug, and
wherein said pharmaceutically acceptable cation acceptor is contained in an amount of 0.1 to 2 mole equivalent per mole of acidic groups in the acidic drug, and is selected from the group consisting of amines, amino acids, pharmaceutically acceptable metallic salts of weak acids having at least one of an acetate and a citrate, and mixtures thereof, and a mixture of pharmaceutically acceptable basic compounds and said pharmaceutically acceptable metallic salts of weak acids having at least one of an acetate and a citrate, amino acids or the amines.

2. The pharmaceutical preparation according to claim 1, wherein the hardly water-soluble acidic drug is selected from the group consisting of naproxen (C₁₄H₁₄O₃, M.W 230.26), r,s-ibuprofen (C₁₃H₁₈O₂, M.W 206.28), dexibuprofen (S-Ibuprofen, C₁₃H₁₈O₂, M.W 206.28), indomethacin (C₁₉H₁₆ClNO₄, M.W 357.79), acetaminophen (M.W 151.17), mefenamic acid (C₁₅H₁₅NO₂, M.W 241.29), chlorocinnazine hydrochloride (C₂₆H₂₇N₂C₁₋₂HCl, MW: 475.88), loxoprofen (C₁₅H₁₈O₃, MW: 246.31), fenoprofen (C₁₅H₁₄O₃, MW: 242.27), ketoprofen (C₁₆H₁₄O₃, MW: 254.29), pranoprofen (C₁₅H₁₃NO₃, MW: 255.27), meclofenamic acid (C₁₄H₁₁Cl₂NO₂, MW: 296.15) and salts thereof, sulindac (C₂₀H₁₇FO₃S, MW: 356.42), piroxicam (C₁₅H₁₃N₃O₄S, MW: 331.35), meloxicam (C₁₄H₁₃N₃O₄S₂, MW: 351.41), tenoxicam (C₁₃H₁₁N₃O₄S₂, MW: 337.38), diclofenac (C₁₄H₁₁Cl₂NO₂, MW: 296.15), aceclofenac (C₁₆H₁₃Cl₂NO₄, MW: 354.19), rebamipide (C₁₉H₁₅ClN₂O₄, MW: 370.79), enalapril maleate(C₂₀H₂₈N₂O₅, MW: 492.52), captopril (C₉H₁₅NO₃S, MW: 217.29), ramipril (C₂₃H₃₂N₂O₅ MW: 416.52), fosinopril (C₃₀H₄₆NO₇P, MW: 563.67), benazepril (C₂₄H₂₈N₂O₅, MW: 424.50), quinapril hydrochloride
(C₂₅H₃₀N₂O₅HCl, MW: 474.99), temocapril (C₂₃H₂₈N₂O₅S₂ MW: 476.62), cilazapril (C₂₂H₃₁N₃O₅ MW: 417.51), lisinopril (C₂₁H₃₁N₃O₅, MW: 405.50), valsartan (C₂₄H₂₉N₅O₃, MW: 435.53), losartan potassium (C₂₂H₂₂ClKN₆O MW: 461.01), irbesartan (C₂₅H₂₈N₆O MW: 428.54), cetirizine hydrochloride (C₂₁H₂₅ClN₂O₃, MW: 388.90), diphenhydramine hydrochloride (C₁₇H₂₁NO. HCl, MW: 291.82), fexofenadine (C₃₂H₃₉NO₄, MW: 501.67), pseudoephedrine hydrochloride (C₁₀H₁₅NO HCl, MW: 201.70), methylephedrine hydrorchloride (C₁₁H₁₇NO.HCl, MW: 215.72), dextromethorphan hydrobromide (C₁₈H₂₅NO HBr H₂O, MW: 370.33), guaifenesin (C₁₀H₁₄O₄, MW: 198.22), noscapine (C₂₂H₂₃NO₇, MW: 413.43), tri-metoquinol hydrocloride (C₁₉H₂₃NO₅. HCl, MW: 399.87), doxylamine succinate (C₁₇H₂₂N₂O, C₄H₆O₄, MW: 388.5), ambroxol (C₁₃H₁₈Br₂N₂O, MW: 378.11), letosteine (C₁₀H₁₇NO₄S₂, MW: 279.37), sobrerol (C₁₀H₁₈O₂, MW: 170.25), bromhexine hydrochloride (C₁₄H₂₀Br₂N₂HCl, MW: 412.59), chlorpheniramine maleate (C₁₆H₁₉ClN₂. C₄H₄O₄, MW: 390.87) and optical isomers thereof.

3. The pharmaceutical preparation according to claim 1, wherein said pharmaceutically acceptable cation acceptor is selected from the group consisting of sodium acetate, potassium acetate, potassium citrate, sodium citrate, prolamine, diethanol amine, mono-ethanol amine, tri-ethanol amine, lysine, methylglucamine and mixtures thereof, and the mixture of 1) the pharmaceutically acceptable basic compounds having one of potassium hydroxide and sodium hydroxide, and 2) at least one of sodium acetate, potassium acetate, potassium citrate, sodium citrate, prolamine, diethanol amine, mono-ethanol amine, tri-ethanol amine, lysine, methylglucamine and mixtures thereof.

4. The pharmaceutical preparation according to claim 1, wherein the surfactant is one selected from the group consisting of reaction products of natural or hydrogenated vegetable oils and ethylene glycol, polyoxyethylene sorbitan fatty acid esters, transesterification products of natural vegetable oil tri-glycerides and polyalkylene polyols, polyoxyethylene fatty acid esters, sorbitan fatty acid esters, propylene glycol mono- and di-fatty acid esters, pharmaceutically acceptable C₁₋₅ alkyl or tetrahydrofurfuryl di- or partial-ether of low molecular mono- or poly-oxy-alkanediols, polyoxyethylene fatty acid ethers, polyoxyethylene-polyoxypropylene copolymers or a mixture of two or more thereof.

5. The pharmaceutical preparation according to claim 6, wherein the surfactant is one selected from the group consisting of CREMOPHOR RH40 (Polyoxyl 40 hydrogenated castor oil), CREMOPHOR EL (Polyoxyl 35 castor oil), LABRASOL (polyethylene glycol caprylate/caprate), TRANSCUTOL (diethylene glycolmono-ethyl ether), TWEEN (polysorbate) 20, 21, 40, 61, 65, 80, 81, 85, 120, POLOXAMER 124, 188, 237, 338, 407 (polyoxyethylene-polyoxypropylene), NIKKOL HCO-40 (polyoxyethylene glycolated natural or hydrogenated castor oil), MYRJ 45 (polyoxyethylene(8)stearate), TAGAT L (polyoxyethylene(30) mono-laurate), MARLOSOL 1820 (polyoxyethylene(20) stearate), MARLOSOL OL 15 (polyoxyethylene(15) oleate), BRIJ 96 (polyoxyethylene(10) oleyl ether), VOLPO 015 (polyoxyethylene(15) oleyl ether), MARLOWET OA30 (polyoxyethylene(30) oley ether), MARLOWET LMA 20 (polyoxyethylene(20) oleyl ether), SYPERONIC PE L44 (polyoxyethylene-polyoxypropylene copolymer), SYPERONIC F127 (polyoxyethylene-polyoxypropylene copolymer, LABRAFIL M 2125 CS (linoleoyl macrogol glycerides), LABRAFAC PG (propylene glycol dicaprylocaprate), Imbitor (caprylic acid/capric acid mono- and di-glyceride), sorbitan mono-stearate, sorbitan tri-stearate, sorbitan mono-oleate, polyethylene glycol mono-oleate, MIGLYOL 840 (propylene glycol dicaprylate), GELUCIR 44/14 (lauroyl polyoxyl-32 glyceride) and the mixtures thereof.

6. The pharmaceutical preparation according to claim 1, wherein the polyethylene glycol has an average molecular weight of 200 to 800.

7. The pharmaceutical preparation according to claim 1, wherein the polyethylene glycol is replaced by one selected from the group consisting of tetraglycol, polyethylene glycol ethers of alcohols and polyethylene glycol copolymers.

8. The pharmaceutical preparation according to claim 1, wherein pH of the solvent system is in the range of 2.0 to 8.0.
